(19) United States
(12) Patent Application Publication
(10) Pub. No.: US 2015/0173843 A1
(43) Pub. Date: Jun. 25, 2015

(54) TRACKING MEDICAL DEVICES

(71) Applicant: DePuy Synthes Products, LLC, Raynham, MA (US)

(72) Inventors: Thomas Maughan, West Chester, PA (US); Frank Wilson, West Chester, PA (US)

(21) Appl. No.: 14/171,002

(22) Filed: Feb. 3, 2014

Related U.S. Application Data
(60) Provisional application No. 61/920,308, filed on Dec. 23, 2013.

(51) Int. Cl. A61B 19/00; A61B 17/86; G06Q 50/24; A61B 17/80

(52) U.S. Cl. CPC A61B 19/44; A61B 17/80; A61B 17/86; G06Q 50/24

(57) ABSTRACT

Techniques for tracking medical devices, for instance medical implants, are described herein. Medical devices can be tracked that are, for example, not directly marked with an identifier. In one embodiment, a device tracking application can execute on a computer system. Such a computer system can include one more computing devices that can optionally communicate with each other to send and receive information associated with medical devices. In accordance with one embodiment, a storage object image representative of a storage object is displayed on a display of a computer system. The storage object image can include a plurality of storage location areas that represent a plurality of storage locations within the storage object. Devices can be tracked based on the storage location areas of the storage object image.

### CROSS REFERENCE TO RELATED APPLICATIONS

This Application claims the benefit of U.S. Provisional Patent Application Ser. No. 61/920,308, filed Dec. 23, 2013, the disclosure of which is hereby incorporated by reference as if set forth in its entirety herein.

### BACKGROUND

Medical devices, such as medical implants, can sometimes be tracked by marking them with a unique device identifier. However, some medical devices are difficult to track. For instance, some medical devices, such as some medical screws for instance, are too small to legibly etch a device identifier onto the device. Other medical devices may not have a surface that can be etched upon. Existing approaches to marking such medical devices include marking the package that contains the medical device. However, existing approaches to marking such medical devices are costly and inefficient. For example, some medical devices may be unpackaged and stored in a storage object prior to being used on a patient. In some cases, once a package has been opened and a medical device has been removed from a package, existing approaches may be unable to efficiently track the unpackaged medical device.

### SUMMARY

Techniques for tracking medical devices, for instance medical implants, are described herein. In one embodiment, one or more device tracking applications can execute on a computer system. Such a computer system can include one more computing devices, such as for example a client device and a server device, that can optionally communicate with each other to send and receive information associated with medical devices. In accordance with one embodiment, a storage object image representative of a storage object is displayed on one or more displays of a computer system. The storage object image can include a plurality of storage location areas that represent a plurality of storage locations within the storage object. The plurality of storage locations can be configured to store a plurality of medical devices. The computer system can receive a first input comprising a first selection of a first storage location area and an indication of a first medical device identifier corresponding to a first medical device. The first input can be associated with a storage of the first medical device in a first storage location represented by the first storage location area. At least one memory of the computer system can store the first medical device identifier and a record of an association between the first medical device identifier and the first storage location. The computer system can receive a second input comprising a second selection of the first storage location area, wherein the second input is associated with a use of the first medical device in connection with a patient. In response to receiving the second input, the computer system can access the first medical device identifier in the at least one memory of the computer system. The computer system may also store a record of an association between the first medical device identifier and the patient. This may allow, for example, a patient upon whom a medical device has been used to be quickly and efficiently determined in scenarios such as product recalls and others.

The foregoing summarizes only a few aspects of the present disclosure and is not intended to be reflective of the full scope of the present disclosure. Additional features and advantages of the disclosure are set forth in the following description, may be apparent from the description, or may be learned by practicing the invention. Moreover, both the foregoing summary and following detailed description are exemplary and explanatory and are intended to provide further explanation of the disclosure.

### BRIEF DESCRIPTION OF THE DRAWINGS

The foregoing summary, as well as the following detailed description of example embodiments of the present disclosure, will be better understood when read in conjunction with the appended drawings. For the purposes of illustrating the example embodiments of the present disclosure, references to the drawings are made. It should be understood, however, that the application is not limited to the precise arrangements and instrumentalities shown. In the drawings:

FIG. 1 is a block diagram of an example computing device for use in accordance with the present disclosure;

FIG. 2 is an example communication architecture for use in accordance with the present disclosure;

FIG. 3 is a perspective view of an example storage object;

FIG. 4 is a perspective view of the storage object of FIG. 3, wherein a cover is removed to show various medical devices stored at various storage locations within the storage object;

FIG. 5 is an exploded view of the storage object of FIG. 3, wherein the medical devices are aligned with their respective storage locations along a transverse direction;

FIG. 6 is a storage object image representative of the storage object shown in FIG. 3 in accordance with an example embodiment, wherein the storage object image includes a plurality of storage location areas that represent a plurality of storage locations within the storage object, the plurality of storage locations configured to store a plurality of medical devices;

FIG. 7 is a perspective view of a medical screw that can be tracked in accordance with an example embodiment;

FIGS. 8A-B are perspective views of respective medical plates that can be tracked in accordance with an example embodiment; and

FIG. 9 is a flowchart of a method for tracking a medical device in accordance with an example embodiment.

### DETAILED DESCRIPTION OF ILLUSTRATIVE EMBODIMENTS

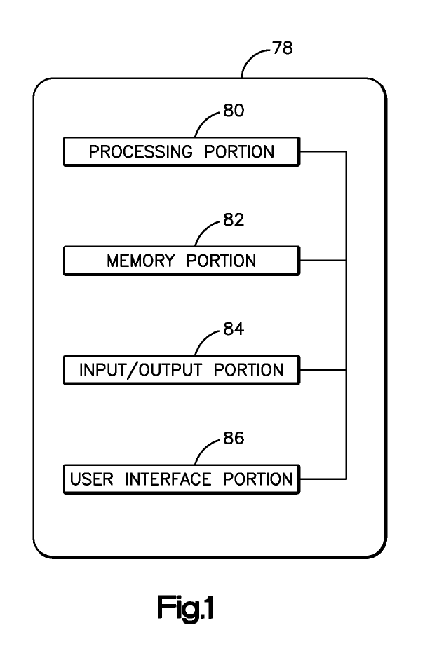

Referring to FIG. 1, a suitable computing device such as example computing device 78 can be configured to host a medical device tracking application. The medical device tracking application is a program, such as software or hardware or a combination of both, that can be run on one or more suitable computing devices. It will be appreciated that the embodiments described herein can be applied to track any medical device, for instance any medical implant. In this regard, reference below to the medical device tracking application can be further construed as an application that can assist in the tracking of a medical device while it is stored within a storage object, and can additionally assist in the tracking of a medical device after it is used on a patient. It will be understood that the computing device 78 can include any appropriate device, examples of which include a desktop computing device, a server computing device, or a portable computing device, such as a laptop, tablet, or smart phone.

In an example configuration, the computing device 78 includes a processing portion 80, a memory portion 82, an input/output portion 84, and a user interface (UI) portion 86. It is emphasized that the block diagram depiction of the computing device 78 is exemplary and not intended to imply a specific implementation and/or configuration. The processing portion 80, memory portion 82, input/output portion 84, and user interface portion 86 can be coupled together to allow communications therebetween. As should be appreciated, any of the above components may be distributed across one or more separate devices and/or locations.

In various embodiments, the input/output portion 84 includes a receiver of the computing device 78, a transmitter of the computing device 78, or a combination thereof. The input/output portion 84 is capable of receiving and/or providing information pertaining to communicate a network such as, for example, the Internet. As should be appreciated, transmit and receive functionality may also be provided by one or more devices external to the computing device 78.

The processing portion 80 may include one or more processors. Depending upon the exact configuration and type of processor, the memory portion 82 can be volatile (such as some types of RAM), non-volatile (such as ROM, flash memory, etc.), or a combination thereof. The computing device 78 can include additional storage (e.g., removable storage and/or non-removable storage) including, but not limited to, tape, flash memory, smart cards, CD-ROM, digital versatile disks (DVD) or other optical storage, magnetic cassettes, magnetic tape, magnetic disk storage or other magnetic storage devices, universal serial bus (USB) compatible memory, or any other medium which can be used to store information and which can be accessed by the computing device 78.

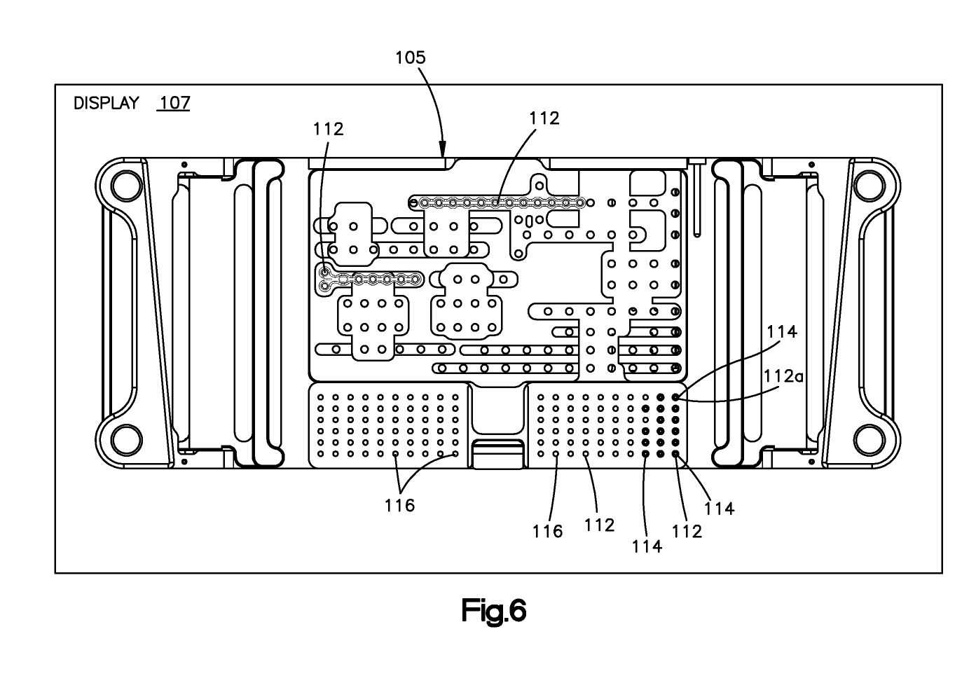

The computing device 78 also can contain the user interface portion 86 allowing a user to communicate with the computing device 78. The user interface 86 can include inputs that provide the ability to control the computing device 78, via, for example, buttons, soft keys, a mouse, voice actuated controls, a touch screen, movement of the computing device 78, visual cues (e.g., moving a hand in front of a camera on the computing device 78), or the like. The user interface portion may also include, for example, a scanner for scanning of information such as bar codes. The user interface portion 86 can provide outputs, including visual information (e.g., via a display), audio information (e.g., via speaker), mechanically (e.g., via a vibrating mechanism), or a combination thereof. In various configurations, the user interface portion 86 can include a display, a touch screen, a keyboard, a mouse, an accelerometer, a motion detector, a speaker, a microphone, a camera, a tilt sensor, or any combination thereof. The user interface portion 86 can further include any suitable device for inputting biometric information, such as, for example, fingerprint information, retinal information, voice information, and/or facial characteristic information. Thus, a computer system including, for example, computing device 78 can include a processor, a display coupled to the processor, and a memory in communication with the processor. The memory can have stored therein instructions that, upon execution by the processor, cause the computer system to perform operations, such as the operations described below. The display can be configured to display visual information, such as described with reference to FIG. 6. As used herein, the term computer system can refer to a system that includes one or more computing devices 78. For instance, the computer system can include one or more server computing devices that communicate with one or more client computing devices.

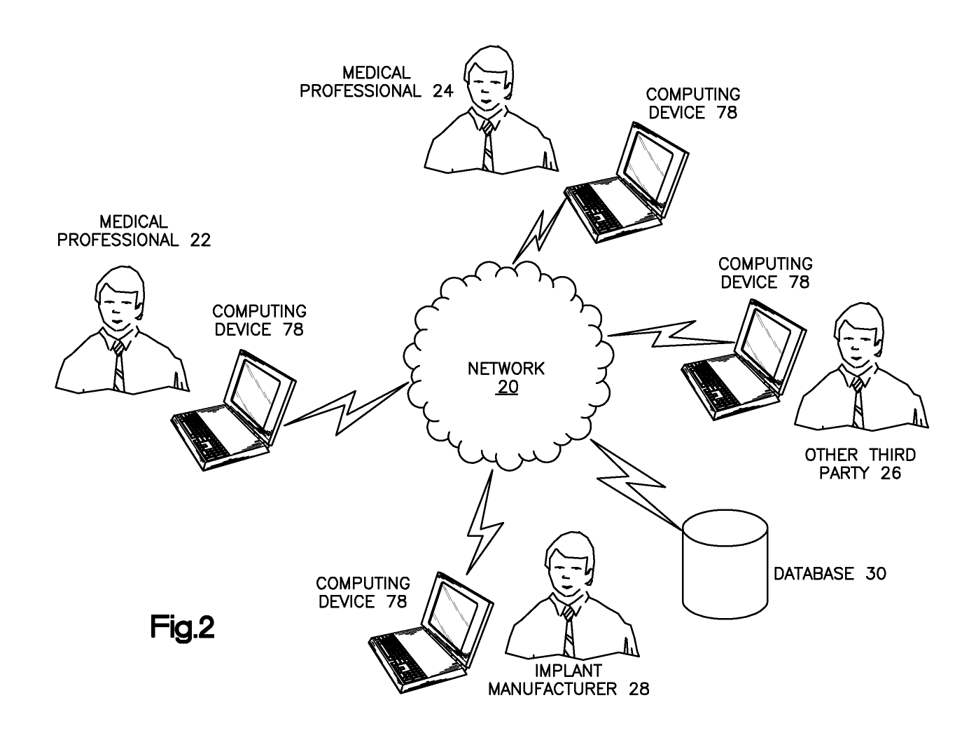

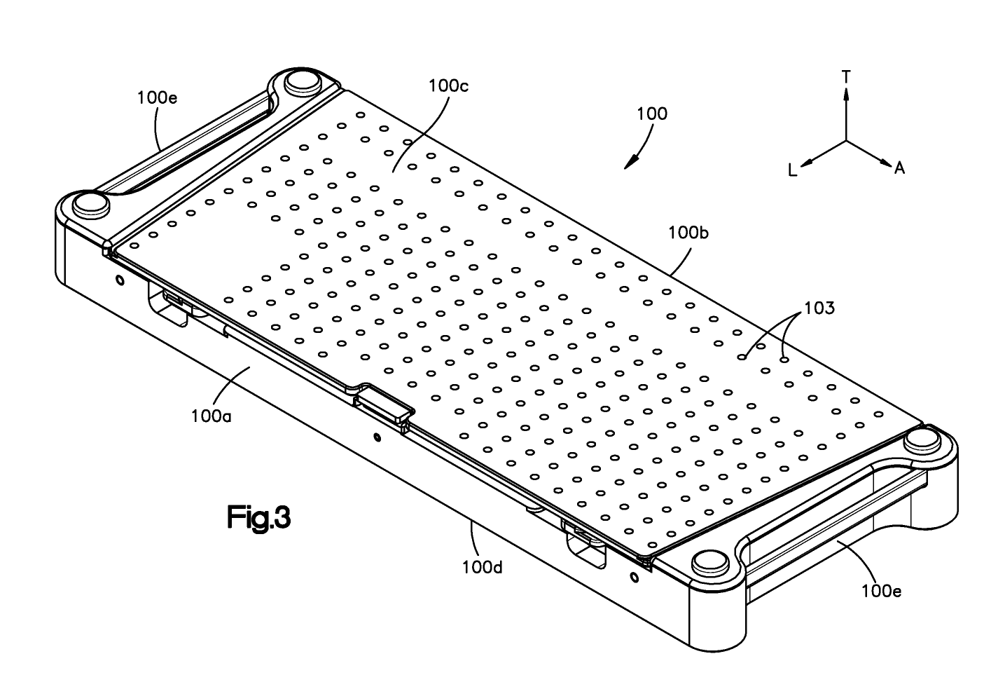

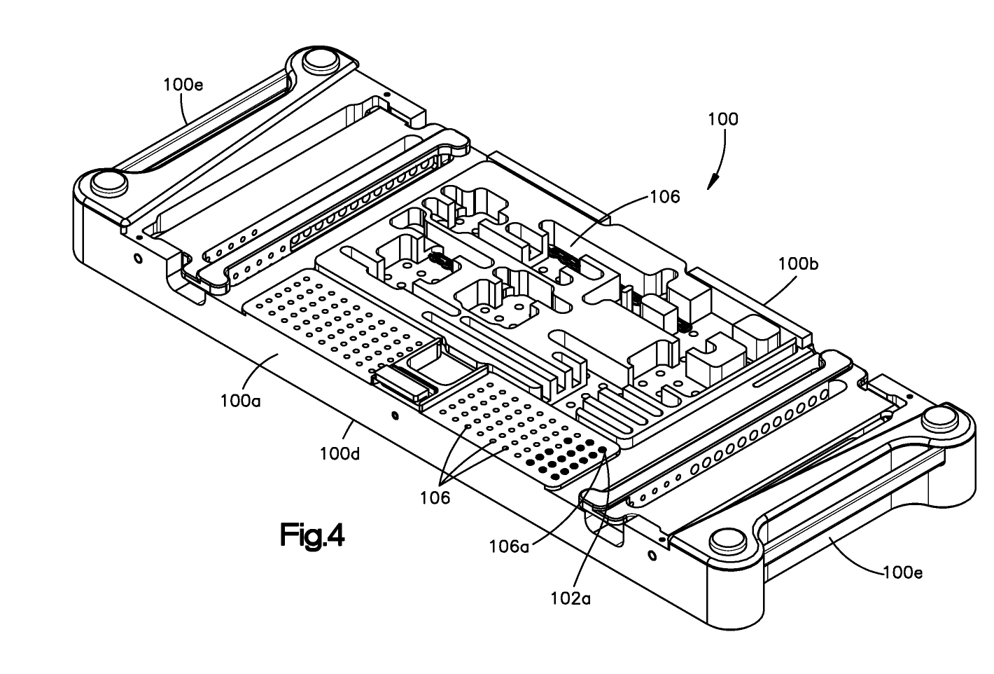

FIG. 2 depicts one example of a suitable communication architecture that can facilitate the tracking of medical devices, it being appreciated that numerous suitable alternative communication architectures are envisioned. Once one or more medical device tracking applications have been installed onto a computer system including, for example, the computing device 78 such as described above and/or possibly other devices, information may be transferred between other computing devices 78 on a common network 20, such as, for example, the Internet. Example computing devices 78 include, without limitation, desktops, laptops, mobile phones, tablet computers, or the like. In an example configuration, referring also to FIGS. 3-5, a first medical professional 22 and/or a device manufacturer such as an implant manufacturer 28 may store medical devices 102 within a storage object 100. When one of the medical devices 102 are stored, a storage location area (via the device tracking application) can be selected that corresponds to a location within the storage object 100 where the one medical device 102 is stored. Thus, as further described below, the medical device 102 and information associated with the medical device 102 can be tracked according to its location within the storage object 100, thereby enabling the tracking of medical devices that do not display an identifier or are otherwise not directly marked. This information can be transmitted to other computing devices 78, such as between mobile devices for example, via the network 20. For instance, a second medical professional 24 or a third party 26 may receive the information associated with the medical device 102 via the network 20.

In an example embodiment, using the device tracking application as described herein, the second medical professional 24 can select the storage location area when the medical device 102 is used in connection with a patient, thereby discovering the information associated with the medical device 102 that is being used by the patient. Thus, medical devices 102 can be tracked from the time that they are stored in the storage object 100 until after they are used in patients. Alternatively, the medical professional 22 or other party that stores the medical device within the storage object can also use the medical device on the patient. After the medical device is stored within the storage object, information related to the medical device can be stored in the database 30 and/or accessed by the computing devices 78. Medical devices 102 can also be referred to herein as inventory, without limitation.

With continuing reference to FIGS. 3-5, while the storage object 100 is illustrated as a graphic case, it will be appreciated that devices can be tracked that are stored within any type of unit as desired. Thus, it will be understood that the storage object 100 can be implemented by, for example, a graphic case, a screw rack, a removable module that can be stored within a graphic case, or the like, or any appropriate combination thereof.

The computing devices 78 and the database 30 depicted in FIG. 2 can be operated in whole or in part by, for example, a medical device manufacturing company, a hospital, a healthcare professional, another third party, or by any combination of any of the above entities. As should be appreciated, each of the parties set forth above and/or other relevant parties may operate any number of respective computers and may communicate internally and externally using any number of networks including, for example, wide area networks (WAN's) such as the Internet or local area networks (LAN's). Database 30 may be used, for example, to store information medical devices that are used within patients or stored within a storage object. Database 30 may also be used, for example, to store information obtained from parties such as healthcare professionals and medical device manufacturers.

Referring still to FIGS. 3-5, the example storage object 100 can define a front end 100a and an opposed rear end 100b that is spaced from the front end 100a along a longitudinal direction L, a top end 100c and an opposed bottom end 100d that is spaced from the top end 100c along a transverse direction T that is substantially perpendicular to the longitudinal direction L, and opposed sides 100e that are spaced from each other along a lateral direction A that is perpendicular to both the transverse direction T and the longitudinal direction L. Unless otherwise indicated herein, the terms “lateral,” “longitudinal,” and “transverse” are used to describe the orthogonal directional components of various components. The terms “inboard” and “inner,” and “outboard” and “outer” and like terms when used with respect to a specified directional component are intended to refer to directions along the directional component toward and away from the center of the apparatus being described.

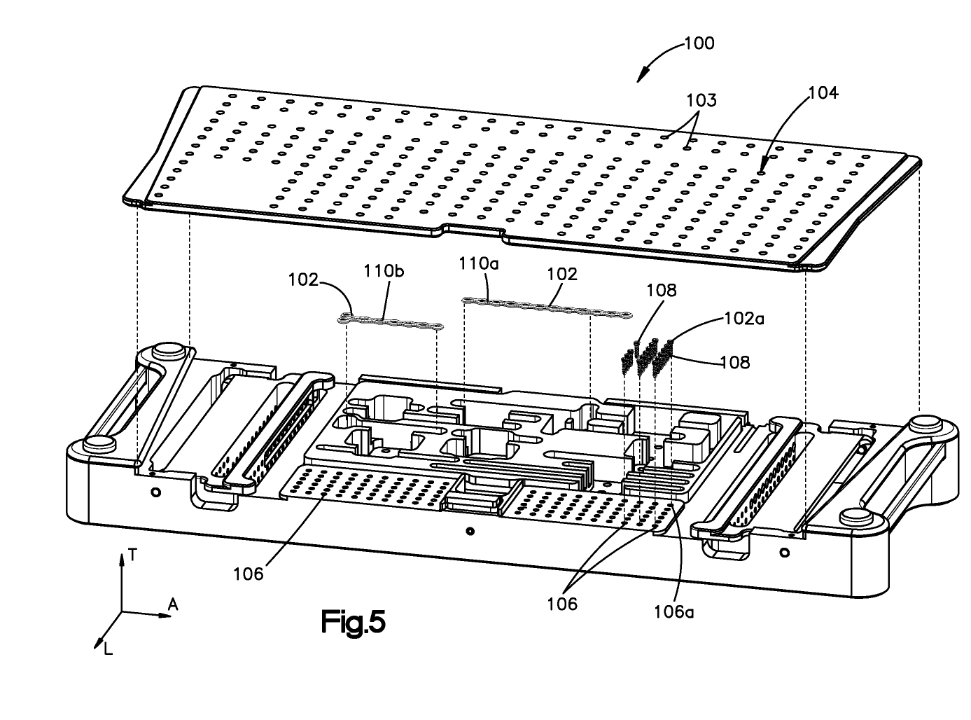

As will be appreciated from the description below, the top end 100c can define a cover 104 that can be removed or opened so that medical devices 102 can be placed in the storage object 100 or removed from the storage object 100. It will further be appreciated that the storage object 100 can alternatively be accessed so as to place medical devices within the storage object 100 or to remove medical devices from the storage object 100. As illustrated, the cover 104 can include holes 103 that allow steam to pass through the cover 104 so as to sterilize the medical devices 102 that are stored within the storage object 100. It will be appreciated that the holes 103 can be alternatively placed as desired, for instance at the bottom end 100d. Referring in particular to FIG. 5, in accordance with the illustrated example, medical devices 102 can be aligned with respective storage locations 106 along the transverse direction T so that the storage locations 106 can receive the respective medical devices 100 along the transverse direction T. It should be appreciated that while the longitudinal and lateral directions L and A are illustrated as extending along a horizontal plane, and that the transverse direction T is illustrated as extending along a vertical plane, the planes that encompass the various directions may differ during use, depending, for instance, on the orientation of the various components.

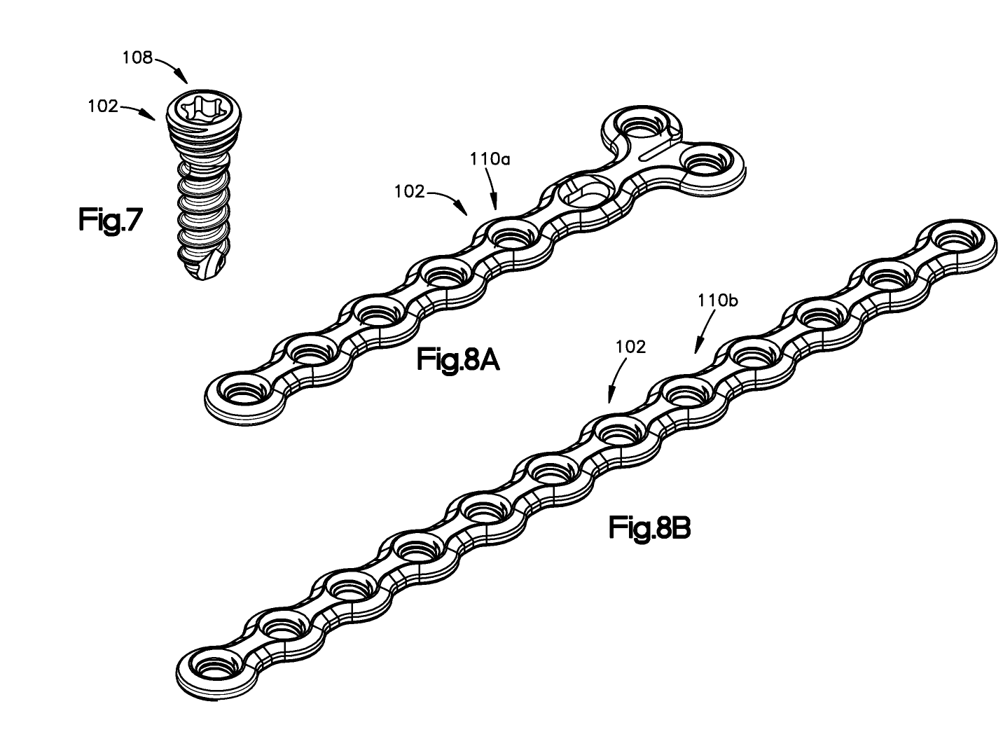

It will be understood that the medical devices 102 can include any medical device as desired. For instance, referring also to FIGS. 7-8B, the medical devices 102 that can be stored within the storage object 100, and thus can be tracked by the device tracking application, can include, for example, medical screws 108 and medical plates 110a and 110b. The medical devices 102 can be, for example, implanted within a given patient. The medical devices 102 can also be, for example, directly or indirectly attached and/or fixated to the patient. The medical devices 102 may also include any other type of device that can be used for medical purposes in connection with one or more patients. The medical devices 102 can be sterile medical devices or nonsterile medical devices. The medical devices 102 can include medical implants that can be fabricated from any biocompatible, implantable material as desired, including metals such as titanium, titanium alloy such as Ti-6Al-7Nb, or stainless steel, polymers such as polyetheretherketone (PEEK), reinforced plastics, and the like. In some cases, the medical devices 102 may be directly marked with corresponding medical device identifiers. For example, in some cases, a particular medical device 102 may have a corresponding medical device identifier etched onto its surface. However, the device tracking application disclosed herein may also be used to track medical devices 102 that are not directly marked. Thus, in some cases, medical devices 102 may not be directly marked with corresponding medical device identifiers. For example, in some cases, a medical device identifier may be provided on a device package as opposed to being directly displayed on the corresponding medical device itself. As should also be appreciated, in some cases, the medical devices 102 may include any combination of the different devices and device attributes described above.

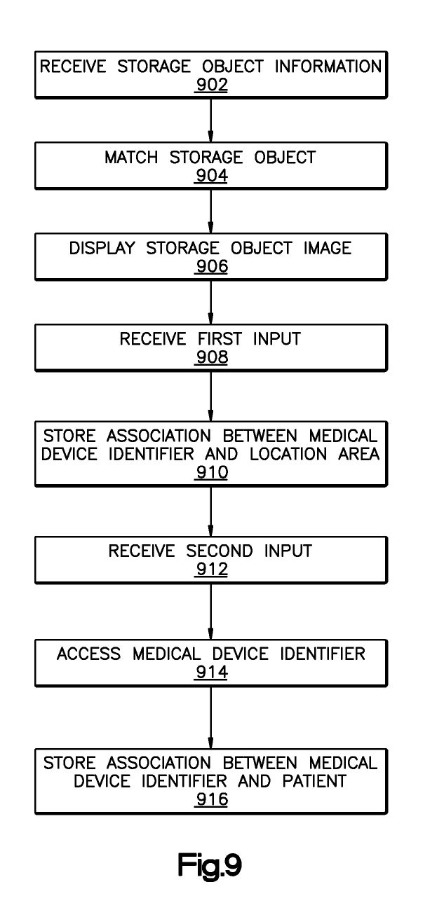

Referring to FIG. 9, the steps depicted in FIG. 9 can be performed by one or more medical device tracking applications, which can be installed on a computer system that includes one or computing devices such as a desktop, laptop, mobile phone, or a tablet computer. Components or computing devices within the computer system can communicate with each other via an architecture as depicted in FIG. 2 or an alternatively configured architecture as desired. In some cases, different computers and/or computing devices within a computer system may perform various different steps depicted in FIG. 9.

With continuing reference to FIG. 9, storage object information is received at 902. The received storage object information can identify a storage object such as the example storage object 100. For instance, the storage object 100 can include an identifier, such as a serial number for instance. It will be understood that storage objects may be owned by a hospital or by another party or entity, and storage objects may or may be not be identified by serial numbers. In accordance with an example embodiment, the computer system can receive an identifier corresponding to the storage object 100. At 904, in accordance with the illustrated embodiment, the computer system matches the storage object 100 to a storage object image 105. For instance, the storage object 100 can include a bar code in an example embodiment, and a graphic, for instance the storage object image 105, can be displayed when the bar code is scanned. In another example embodiment, the computer system can store and/or access a file that specifies one or more storage object identifiers. For each storage object identifier, the computer system can list one or more storage object images that are associated with the identifier. The computer system may then, for example, match a particular storage object to the one or more respective storage object images listed in the file.

Alternatively or additionally, at 902, the computer system can receive a photographic image of the storage object 100. In an example embodiment, based on the photographic image, the computer system can recognize and identify a particular storage object from a set of available storage objects. Further, at 904, the computer system can match the particular storage object to a particular storage object image. In some cases, multiple available storage object images may be stored by the computer system and presented, upon request, to a user. The user may then, for example, match the photographic image of a particular storage object to one or more of the stored available storage object images. In some cases, the user may match the photographic image to a stored image based on visual cues such as, for example, a shape and/or color of the storage object, a number and/or position of storage locations and storage location areas and the like. Also, in some cases, photo and/or image recognition software may be employed by a computer to recognize one or more shapes, patterns or other items within a storage object photograph and match the storage object photograph to one or more stored available storage object images. As yet another example, the computer system can create a new storage object image based on a photograph or other input that describes a respective storage object. For example, in some cases, photo and/or image recognition software may generate a new storage object image by determining, for example, a shape of a storage object and positions of various storage locations within the storage object based on a scanned photograph. As another example, a user may provide input to identify, for example, a shape of a storage object and positions of various storage locations within the storage object.

Identifiers, such as serial numbers for example, may be assigned to respective storage objects by the manufacturers of the respective storage objects. In some cases, the identifiers assigned by a manufacturer may be unique identifiers. Alternatively or additionally, a unique identifier may be assigned to a particular storage object by a user and/or by the device tracking application. In some cases, the unique identifier can be stored by the device tracking application. The unique identifier of a particular storage object can be displayed on the particular storage object. The unique identifiers can, for example, be particularly useful in order to differentiate between storage objects that are the same model as one another or that may otherwise have a similar size, shape and/or appearance to one another. Thus, unique identifiers may be helpful, for example, to differentiate storage objects from each other in scenarios in which a hospital has multiple storage objects that are similar to each other. Differentiating similar storage objects from each other can be useful in tracking medical devices that are tracked according to their respective locations in a particular storage object, as described further below. As should be appreciated, however, storage objects need not necessarily be assigned unique identifiers in order to perform any or all of the tracking techniques described herein.

Referring also to FIG. 6, in accordance with the illustrated embodiment, at 906 the computer system, and in particular a computer display 107 of the computer system, can display a storage object image such as the example storage object image 105 that is representative of a storage object, for instance the example storage object 100. The display 107 can include inputs, such as a touch screen for example, that provide the ability to control the computer system. Input may also be provided, for example, via other inputs of a computing device such as mouse, keyboard, camera, voice detection and the like. The display 107 can provide visual information, such as the example storage object image 105, to a user. The storage object image 105 can include a plurality of storage location areas 112 that represent a plurality of the storage locations 106 within the storage object 100. The plurality of storage locations 106 can be configured to store a plurality of medical devices, such as sterile or nonsterile medical screws, medical plates, other medical implants, other medical devices attached or fixated to a patient, other assorted medical devices, or any combination thereof. For instance, in accordance with the example illustration, the storage locations 106 can define recesses or holes that are sized so as to receive at least a portion of select medical devices 102 so as to secure the medical devices 102 within the storage object 100, although it will be understood that the storage locations 106 can be alternatively shaped as desired.

In accordance with an example embodiment, a user can select one of the storage location areas 112 (e.g., first storage location area 112a) on the storage object image 105 when one of the medical devices 102 (e.g., first medical device 102a) is stored within the storage object 100 at one of the storage locations 106 (e.g., first storage location 106a that corresponds to the first storage location area 112a). As further described below, by selecting the first storage location area 112a that corresponds to the first storage location 106a at which the first medical device 102a is to be stored, information corresponding to the first medical device 102a can be associated and tracked with respect to the associated first storage location area 112a. It will be understood that users can include the medical professional 22, the medical professional 24, a device manufacturer such as the implant manufacturer 28, or the other third party 26 as described above with respect to FIG. 2. Further, selections can be made by users via a variety of mechanisms including, for example, inputs provided by user interface 86 of the example computing device 78. For instance, users can select a given storage location area 112 via a touch-screen display, such as the display 107 for example, by touching a portion of the display 107 that corresponds to the given storage location area 112. Alternatively, users can use a mouse or keyboard to provide inputs and selections to the device tracking application. As yet another example, users can perform one or more gestures to select a given storage location area 112 such as, for example, pointing at and/or hovering over the given storage location area 112.

At 908, when a user selection is made, the computer system, and in particular the device tracking application, receives an input, for instance a first input. The first input can include the user selection as described above, which can be referred to as a first selection. The first input can identify the first storage location area 112a from the plurality of storage location areas 112. The first storage location area 112a may represent a first storage location 106a at which one of the medical devices 102, for instance a first medical device 102a, is to be stored. The first input may further include an indication of a medical device identifier corresponding to the first medical device 102a, which can also be referred to as a first medical device identifier. A medical device identifier may be any identifier that can be used to identify a corresponding medical device 102. A medical device identifier may be, for example, a unique identifier. However, it is not required that a medical device identifier must be a unique identifier. In some cases, a medical device identifier may be, for example, a global trade identification number (GTIN). A medical device identifier can, for example, be scanned from a bar code. A medical device identifier can also, for example, be disposed on a label of a package that contains a corresponding medical device 102. As another example, the a medical device identifier can be etched or otherwise directly disposed on a corresponding medical device 102. It will be understood that a medical device identifier can be alternatively provided to the computer system as desired.

Additionally, for example, while the first medical device 102a is illustrated as a screw, it will further be understood that the first medical device 102a can be any device as desired such as, for example, a plate or another medical device. Thus, in accordance with the example embodiment, the first input is associated with a storage of the first medical device 102a at the first storage location 106a represented by the first storage location area 112a. Further, for example, the device tracking application disclosed herein may, in some cases, enable the first medical device 102a to be tracked with reference to the first storage location 106a even in cases when a corresponding medical device identifier is not directly etched onto or otherwise directly displayed on the device itself.

At 910, a memory of the computer system can store the first medical device identifier and a record of an association between the first medical device identifier and the first storage location 106a. As set forth above, the first medical device identifier is a medical device identifier that corresponds to the first medical device. In some cases, the stored record may associate the first medical device identifier with the first storage location 106a based on an identifier or other indication of the first storage location 106a and/or the first storage location area 112a (which corresponds the first storage location 106a). Also, in some cases, the stored record may also associate the first medical device identifier and/or the first storage location 106a with information that identifies a particular storage object 100 (e.g., a unique identifier assigned to storage object 100) that includes the first storage location 106a. For instance, the record of the association between the first medical device identifier, the first storage location 106a, and, in some cases, the particular storage object 100, can be stored in a hospital inventory database, or alternatively stored as desired. Further, the computer system can display the first medical device identifier adjacent to the first storage location area 112a. Thus, by viewing the storage object image 105, users can identify that the first medical device 102a having the first medical device identifier is stored at the first storage location 106a that is represented by the first storage location area 112a. It will be understood that medical device identifiers may be alternatively displayed as desired such that their respective storage locations can be determined by users. For instance, the computer system can be configured so as to display a select medical device identifier when a cursor rolls over its corresponding storage location area 112 on the storage object image 105. Further, the memory, for instance the database 30 or a hospital inventory database, can be searched to access the record of the association between the first medical device identifier and the first storage location 106a and, in some cases, the particular storage device 100. In an example scenario, a user may determine the storage location 106 of a particular medical device 102 by providing the medical device identifier of the medical device to the device tracking application. Based on the medical device identifier of the medical device, the device tracking application can display various information associated with the medical device. For instance, based on the medical device identifier, the device tracking application can display various information relative to the location of the medical device. For instance, in accordance with various embodiments, the computer system, and in particular the device tracking application, can display the identifier of the storage object in which the device is stored, the storage location (within the storage object) at which the medical device is stored, the location (e.g., within a hospital) where the storage object is located, or the like, or a combination thereof. For example, in some cases, an identifier of a hospital and/or doctor that possesses the first medical device or other information may also be associated and stored with the first medical device identifier and the first storage location.

With continuing reference to FIG. 9, in accordance with an example embodiment, steps 906 and 908 may be performed in a different setting with different computing devices as compared to the setting in which step 912 is performed. In some cases, steps 906 and 908 may, for example, be performed in a storage/inventory type of setting, while step 912 may be performed in an operating room or other type of patient-procedure setting. For example, prior to performance of step 912, the storage object can be physically moved from a storage/inventory area into an operating room. In accordance with another example embodiment, step 906 may sometimes be repeated prior to performing step 912. For example, in some cases, step 906 can be repeated on an operating room computer prior to surgery or another procedure. Thus, for example, in the operating room, a doctor or another medical professional could enter an identifier of the storage object into a computer and a respective storage object image could be retrieved from memory and displayed on the operating room computer.

In accordance with an example embodiment, users can select a particular storage location area 112 on the storage object image 105 when the medical device 102 that is stored at a particular storage location 106 that is represented by the particular storage location area 112 is used on a patient (such as, for example, in an operating room or another patient-procedure setting). By doing so, the computer system can identify a particular medical device 102 that is being used on a particular patient so that, for example, the medical device 102 can be tracked after use on the patient. Referring again to FIG. 9, in accordance with the illustrated embodiment, the computer system receives a second input at 912. The second input may include, for example, a selection of a particular storage location area 112 such as the first storage location area 112a. The selection of a particular storage location 112 in the second input is referred to herein as a second selection of a particular storage location 112. The second input can be received, for example, when the first medical device 102a is removed from the storage object 100 (and, more specifically, is removed from the first storage location 106a) to be used on a patient. Thus, the second input can include selecting one of the storage location areas 112. A storage location area 112 can be selected in the second input using some or all of the same techniques that can be used to select a storage location area 112 included in the first input. For instance, users can select the storage location area 112 for the second input via a touch-screen display by touching a portion of the display that corresponds to the storage location area 112. Alternatively, users can use a mouse or keyboard to provide inputs and selections to the device tracking application. As yet another example, users can perform one or more gestures to select a given storage location area 112 such as, for example, pointing at and/or hovering over the given storage location area 112. The second input that is received by the computer system can further include an identifier of the patient who is receiving the first medical device 102a that was stored at the first storage location area 112a. Such a patient identifier may include, for example, a name, address, insurance information and/or social security number. The second input can also include, for example, an indication of a time, date, location, hospital, doctor, and other staff that may be associated with an implantation, attachment or other use of the medical device on the patient. Thus, the second input can be associated with a use of the first medical device 102a in connection with the patient.

In response to receiving the second input, the computer system can access the first medical device identifier in the memory of the computer system at 914. For example, the computer system can access the first medical device identifier associated with the first medical device 102a by retrieving and/or identifying the first medical device identifier such that the first medical device identifier can be associated with additional information such as patient information. Also, in some cases, the first medical device identifier may be retrieved and/or identified for display on a display, such as the display 107 for example, of the computer system. At 916, the computer system can also store a record of an association between the first medical device identifier and the patient. Thus, the memory of the computer system, which can be included as part of a hospital inventory database for example, can, in some cases, be searched using an identity (e.g., name, social security number, or the like) of the patient and/or using the medical device identifier of the medical device. By way of example, the device tracking application can receive a patient's name, and can access a record of the patient. Thus, the device tracking application can, in some cases, identify any medical devices that have been used on the patient based on the stored record of the association between the medical device identifier and the patient. Similarly, by way of another example, if the patient has a problem with a medical device that is implanted within or attached to the patient, then a memory (e.g., a database) of the computer system executing the device tracking application can, in some cases, be accessed to identify the one or more devices that have been implanted within or attached to the patient. Thus, the problematic device can be identified. By way of yet another example, if a given device is determined to be problematic and there is a product recall associated with the given device, then the device tracking application can determine which patients the recalled device has been used upon. In some cases, this can be determined because the record of the association between the medical device identifier and the patient is stored at 916.

Referring in particular to FIG. 6, it will be understood that, in some example scenarios, one of the medical devices 102 can be stored in at least one of the storage locations 106, and none of the medical devices 102 can be stored in at least one of the storage locations 106. Further, the computer system can display a graphic indication, for instance a first indication 114, that is associated with each of the storage location areas 112 representing a storage location in which a medical device 102 is stored. The computer system can display a graphic indication, for instance a second indication 116, that is associated with each of the storage location areas representing a storage location in which no medical device is stored. As illustrated, the first indication can be different from the second indication. By way of example, the graphic indications can include a first color when a medical device is stored at the storage location 106 represented by the storage location area 112, and a second color when no medical device is stored at the storage location 106 represented by the storage location area 112. For example, in FIG. 6, a black shaded circle is an example first indication 114 used to represent some storage location areas 112 associated with a storage location 106 in which a medical device 102 is stored. Also, in FIG. 6, a white circle is an example second indication 116 used to represent some storage location areas 112 associated with a storage location 106 in which no medical device 102 is stored. Further, in response to receiving the second input, the computer system can remove the first indication associated with the first storage location area 112a and display the second indication associated with the first storage location area 112a, thus indicating that a medical device is no longer stored at the first storage location 106a represented by the first storage location area 112a.

As should be appreciated, in addition to the first medical device 102a, some or all of the steps depicted in FIG. 9 may be repeated any number of times when, for example, additional medical devices are stored in and/or removed from storage object 100. For example, in some cases, when a second medical device is stored in a second storage location, a second storage location area corresponding to the second storage location may be selected and a second medical device identifier corresponding to the second medical device may be provided to the device tracking application. A record of an association between the second medical device identifier and the second storage location may be stored in memory. Also, for example, when the second medical device is used in connection with a patient, the second storage location area may be selected and the second medical device identifier may be accessed from memory. A record of an association between the second medical device identifier and the patient may also be stored.

There is no requirement that only a single medical device can be stored at any particular storage location. For example, in some cases, multiple medical devices may be stored at a particular storage location. In such cases, for example, records of associations of multiple medical device identifiers with the particular storage location may sometimes be stored in memory. Also, there is no requirement that a particular medical device must occupy only a single storage location. For example, in some cases, a particular medical device may occupy or otherwise be stored at multiple storage locations. In such cases, for example, records of associations of a medical device identifier of the particular medical device and the multiple storage locations may sometimes be stored in memory.

By way of example, if the storage object 100 is dropped or the medical devices 102 stored within the storage object 100 are otherwise mixed together, the tracking application can display an indication that the inventory within the storage object 100 has been mixed. Thus, in accordance with an example embodiment, the computer system can receive an input, for instance a third input, that indicates that multiple possible medical devices 102 are associated with the first storage location area 112a. For example, the storage object 100 may store a number of medical devices 102 that have a similar size and shape to each other, and that do not include identifiers that are directly etched on or otherwise indicated on the medical devices themselves. In an example scenario, the storage object 100 may be dropped, thereby causing all of the medical devices that were previously stored in the storage object 100 to fall out of the storage object and be scattered on the floor. In some cases, several of the dropped medical devices may be capable of fitting into the first storage location 106a. Thus, in some cases, it may not be possible to ascertain exactly which medical device was previously stored in the first storage location 106a. In such a scenario, a third input may be provided to a computer system to indicate that the inventory that was stored in the dropped storage object has been mixed.

In some cases, the computer system may be capable of determining which of the dropped medical devices are capable of fitting into, for example, the first storage location 106a. For example, the computer system may have access to a set of stored information that indicates a size and/or shape of various medical devices. Thus, in some cases, when medical device identifiers or other first input associated with the medical devices is received by the computer system, the computer system may be capable of determining the size and/or shape of each medical device stored in the storage object. Additionally, for example, the computer system may have access to another set of stored information that indicates a size and/or shape of various storage locations within a storage object. Thus, in some cases, when a storage object is identified and matched to a storage object image (e.g., based on an identifier or photograph as described above), the computer system may be capable of determining the size and/or shape of one or more storage locations within the identified storage object. The computer system may then, for example, compare the size and/or shape of the stored medical devices against the size and/or shape of the storage locations to determine, for example, which of the stored medical devices are capable of fitting into the first storage location 106a and other storage locations. The computer system can then, for example, responsively store multiple medical device identifiers associated with each of the possible medical devices 102 that are capable of fitting into the first storage location 106a. The computer system can also, for example, store records of associations between the first storage location 106a and the multiple medical device identifiers of the possible medical devices 102 that are capable of fitting into the first storage location 106a.

Even after inventory has been mixed, the medical device tracking application can identify the medical devices that are stored within a given storage object in accordance with an example embodiment. For instance, when a particular device is selected for use on a patient, the tracking application can display information indicative of part and lot information of the devices of a particular type, rather than displaying information indicative of a specific device. As the mixed devices are replaced within the storage object, the number of unknown devices are reduced.

Also, in some cases, the third input can be associated with a mixing of inventory between the storage object 100 and at least one other storage object. In this scenario, the computer system may, for example, determine which stored medical devices from multiple storage objects are capable of being stored in one more particular storage locations in one or more of the storage devices. The computer system can then, for example, responsively store multiple medical device identifiers associated with each of the possible medical devices 102 from the multiple storage objects that are capable of fitting into the one or more particular storage locations.

It will be understood that received inputs can be associated with any information as desired. For instance, the first input can further include at least one of a cost of the first medical device, a type of the first medical device, a model number of the first medical device, or a description of the first medical device. In another embodiment, the computer system can generate a list identifying one more devices capable of being stored at any storage location 106, for instance the first storage location 106a. The list can be generated based on, for example, a set of stored information indicating the size and/or shape of the devices and another set of stored information indicating the size and/or shape of the respective storage locations 106, as set forth above. In some cases, the computer system may use the sets of stored information to compare the size and/or shape of the devices against the size and/or shape of the storage locations to generate the list of devices capable of being stored at a respective storage location. Also, in some cases, the computer system may use the sets of stored information to determine which storage locations 106 a particular medical device 102 is capable of fitting into. For example, a barcode for a particular medical device can be scanned, and, in response to the scanning, certain storage locations areas 112 may be indicated that correspond to the storage locations 106 into which that particular medical device 102 will fit. The indicated storage locations areas 112 may, for example, light up or blink such that they may be distinguished by the user from other non-indicated storage locations areas 112.

By way of example and in accordance with the description above, one of the screws 108 can be placed in a storage object, such as a screw rack or a removable module for example, by first accessing the tracking application. A bar code on the screw rack can be scanned to access an image (graphic) associated with the screw rack. Identifying information that corresponds to the screw can be scanned or entered into the application. For instance, a label of a package that contains the screw can be scanned so that the tracking application can identify the screw. A location on the graphic can be selected, and the screw can be placed in the selected location. Thus, data associated with the screw can be saved and associated with the location where it is stored. A location on the graphic can be selected, for example, via a mouse or via a touch-screen display.

Continuing with the example above, when the screw from the screw rack is used on a patient, the corresponding storage location area on the screw rack graphic can be selected to obtain the correct part information associated with the screw. It will be understood that when the screw is used on a patient, the screw rack in which the screw is stored can be in a sterile field, and a computing device that executes the tracking application can be in the sterile field or nonsterile field as desired. In an example scenario, a medical professional working in a sterile field can identify which screw is being used on the patient by communicating the location in which the screw is stored in the rack to another person who may be operating a computing device in a nonsterile field. In an example embodiment, the computer system displays additional location information, such as coordinates for example, that correspond to each location in which devices are stored. Thus, the person in the nonsterile field can enter the location information (coordinates) in the application, thereby creating a record that a particular screw was used in a particular patient.

In another example scenario, information can be entered into the device tracking application via a computing device that is controllable, at least in part, using gestures, visual cues (e.g., moving a body in front a camera) and/or voice actuated controls. In such a scenario, information can, in some cases, be directly entered into a computing device in a non-sterile field by a medical professional who is working in a sterile field. This may, in some cases, eliminate the need for an additional computer operator to be in the nonsterile field to receive information from the medical professional in the sterile field. For example, in some cases, the first selection of the first storage location area at step 908 and/or the second selection of the first storage location area at step 912 may be performable using gestures, visual cues and/or voice actuated controls. By way of yet another example, in accordance with an embodiment, a medical professional can enter location information corresponding to a medical device via a wearable computing device or component, such as an optical head-mounted display for example. The wearable computing device or component can be sterilized and located in the sterile field. Thus, the medical professional can use the wearable computing device or component to enter location information in the medical device tracking application without leaving the sterile field. It will be understood that the wearable computing device or component can communicate with other computing devices as desired. The entered location information can identify a medical device. The identified medical device can represent the medical device that was stored at the location corresponding to the entered location information. By entering the location information that identifies the medical device, a record can be created that that the medical device was used in a patient. The record that the medical device was used in the patient can be included in patient records, invoices, inventory records, and other documents as desired. It will be understood that the techniques described herein can allow interested parties to track devices that are, for example, implanted in or attached to patients, which can be especially useful in the event of a product recall for example.

As described above, it will be understood that various types of computer systems can perform the described methods. For instance, the computer system can include a client computing device and a server computing device, and the record of the association between the first medical device identifier and the first storage location 106a can be stored by the server computing device. In one embodiment, the storage object image 105 can be displayed by a web browser executing on the client device. Alternatively, the storage object image 105 can be displayed by a mobile application executing on a mobile computing device. Further, it will be understood that at least some of the techniques described herein may be applied to tracking other product families besides medical devices. Embodiments are described herein with reference to medical devices because some of the benefits described herein may be especially apparent in the context of medical devices. For example, medical devices may sometimes include or consist of small components that cannot be directly marked, may sometimes be required to be sterilized, and may sometimes be implanted in or attached to a human.

While example embodiments of devices for executing the disclosed techniques are described herein, the underlying concepts can be applied to any computing device, processor, or system capable of communicating and presenting information as described herein. The various techniques described herein can be implemented in connection with hardware or software or, where appropriate, with a combination of both. Thus, the methods and apparatuses described herein can be implemented, or certain aspects or portions thereof, can take the form of program code (i.e., instructions) embodied in tangible non-transitory storage media, such as floppy diskettes, CD-ROMs, hard drives, or any other machine-readable storage medium (computer-readable storage medium), wherein, when the program code is loaded into and executed by a machine, such as a computer, the machine becomes an apparatus for performing the techniques described herein. In the case of program code execution on programmable computers, the computing device will generally include a processor, a storage medium readable by the processor (including volatile and non-volatile memory and/or storage elements), at least one input device, and at least one output device, for instance a display. The display can be configured to display visual information. For instance, the displayed visual information can include a storage object image representative of a storage object, wherein the storage object image includes a plurality of storage location areas that represent a plurality of storage locations within the storage object. The program(s) can be implemented in assembly or machine language, if desired. The language can be a compiled or interpreted language, and combined with hardware implementations.

The techniques described herein also can be practiced via communications embodied in the form of program code that is transmitted over some transmission medium, such as over electrical wiring or cabling, through fiber optics, or via any other form of transmission. When implemented on a general-purpose processor, the program code combines with the processor to provide a unique apparatus that operates to invoke the functionality described herein. Additionally, any storage techniques used in connection with the techniques described herein can invariably be a combination of hardware and software.

While the techniques described herein can be implemented and have been described in connection with the various embodiments of the various figures, it is to be understood that other similar embodiments can be used or modifications and additions can be made to the described embodiments without deviating therefrom. For example, it should be appreciated that the steps disclosed above can be performed in the order set forth above, or in any other order as desired. Further, one skilled in the art will recognize that the techniques described in the present application may apply to any environment, whether wired or wireless, and may be applied to any number of such devices connected via a communications network and interacting across the network. Therefore, the techniques described herein should not be limited to any single embodiment, but rather should be construed in breadth and scope in accordance with the appended claims.

1. A computer-implemented method comprising:
displaying, on one or more displays of a computer system, a storage object image representative of a storage object, the storage object image including a plurality of storage location areas that represent a plurality of storage locations within the storage object and that are selectable by a user for association with medical device identifiers, the plurality of storage locations configured to store a plurality of medical devices;
receiving first input comprising a first selection of a first storage location area and an indication of a first medical device identifier corresponding to a first medical device that is not marked with the first medical device identifier, wherein the first input is associated with a storage of the first medical device in a first storage location represented by the first storage location area;
storing, in at least one memory of the computer system, the first medical device identifier and a record of an association between the first medical device identifier and the first storage location;
receiving second input comprising a second selection of the first storage location area, wherein the second input is associated with a use of the first medical device in connection with a patient; and
in response to receiving the second input, accessing the first medical device identifier in the at least one memory of the computer system.

2. The method as recited in claim 1, further comprising storing a record of an association between the first medical device identifier and the patient.

3. The method as recited in claim 1, wherein a medical device is stored in at least one of the storage locations, wherein no medical device is stored in at least one of the storage locations, and wherein the method further comprises:
displaying a first indication associated with each of the storage location areas representing a storage location in which a medical device is stored; and
displaying a second indication associated with each of the storage location areas representing a storage location in which no medical device is stored, wherein the first indication is different from the second indication.

4. The method as recited in claim 3, the method further comprising:
in response to receiving the second input, removing a first indication associated with the first storage location area and displaying a second indication associated with the first storage location area.

5. The method as recited in claim 1, further comprising:
receiving a photographic image of the storage object; and
identifying, based on the photographic image, the storage object image from a set of available storage object images.

6. The method as recited in claim 1, wherein the first medical device identifier is scanned from a package label.

7. The method as recited in claim 1, further comprising:
receiving third input indicating that multiple possible medical devices are associated with the first storage location area; and
responsively storing multiple identifiers associated with each of the possible medical devices and records of associations between the multiple identifiers and the first storage location.

8. The method as recited in claim 1, wherein the first input further comprises at least one of a cost of the first medical device, a type of the first medical device, a model number of the first medical device, or a description of the first medical device.

9. The method as recited in claim 1, the method further comprising generating a list identifying one more devices capable of being stored at the first storage location.

10. The method as recited in claim 1, wherein the first medical device is a sterile medical device.

11. The method as recited in claim 1, wherein the record of the association between the first medical device identifier and the first storage location is stored in a hospital inventory database.

12. The method as recited in claim 1, wherein the first medical device is a screw or a plate.

13. A computer system comprising:
one or more processors;
one or more displays configured to display a storage object image representative of a storage object, the storage object image including a plurality of storage location areas that represent a plurality of storage locations within the storage object and that are selectable by a user for association with medical device identifiers, the plurality of storage locations configured to store a plurality of medical devices;
at least one memory having stored therein instructions that, upon execution by the one or more processors, cause the computer system to perform operations comprising:
receiving first input comprising a first selection of a first storage location area and an indication of a first medical device identifier corresponding to a first medical device, wherein the first input is associated with a storage of the first medical device that is not marked with the first medical device identifier, in a first storage location represented by the first storage location area;
storing, in the at least one memory of the computer system, the first medical device identifier and a record of an association between the first medical device identifier and the first storage location;
receiving second input comprising a second selection of the first storage location area, wherein the second input is associated with a use of the first medical device in connection with a patient; and
in response to receiving the second input, accessing the first medical device identifier in the at least one memory of the computer system.

14. The computer system as recited in claim 13, the at least one memory having stored therein further instructions that, upon execution by the one or more processors, cause the computer system to perform operations further comprising storing a record of an association between the first medical device identifier and the patient.

15. The computer system as recited in claim 13, wherein the computer system comprises a computing device that is controllable using gestures, wherein at least one of the first selection of the first storage location area and the second selection of the first storage location area are performable using gestures that control the computing device.

16. The computer system as recited in claim 13, wherein the computer system comprises a client computing device and a server computing device.

17. One or more non-transitory computer-readable storage media having collectively stored thereon instructions that, upon execution by one or more processors of a computer system, cause the computer system to at least:
display, on one or more displays of the computer system, a storage object image representative of a storage object, the storage object image including a plurality of storage location areas that represent a plurality of storage locations within the storage object and that are selectable by a user for association with medical device identifiers, the plurality of storage locations configured to store a plurality of medical devices;
receive first input comprising a first selection of a first storage location area and an indication of a first medical device identifier corresponding to a first medical device that is not marked with the first medical device identifier, wherein the first input is associated with a storage of the first medical device in a first storage location represented by the first storage location area;
store, in at least one memory of the computer system, the first medical device identifier and a record of an association between the first medical device identifier and the first storage location;
receive second input comprising a second selection of the first storage location area, wherein the second input is associated with a use of the first medical device in connection with a patient; and
in response to receiving the second input, access the first medical device identifier in the at least one memory of the computer system.

18. The non-transitory computer-readable storage media as recited in claim 17, having further stored thereon instructions that, upon execution by the one or more processors of the computer system, cause the computer system to at least store a record of an association between the first medical device identifier and the patient.

19. The non-transitory computer-readable storage media as recited in claim 17, wherein a medical device is stored in at least one of the storage locations, wherein no medical device is stored in at least one of the storage locations, and the non-transitory computer-readable storage media having further stored thereon instructions that, upon execution by the one or more processors of the computer system, cause the computer system to at least:
display a first indication associated with each of the storage location areas representing a storage location in which a medical device is stored; and
display a second indication associated with each of the storage location areas representing a storage location in which no medical device is stored, wherein the first indication is different from the second indication.

20. The non-transitory computer-readable storage media as recited in claim 19, having further stored thereon instructions that, upon execution by the one or more processors of the computer system, cause the computer system to at least:
in response to receiving the second input, remove a first indication associated with the first storage location area and display a second indication associated with the first storage location area.
